(19) United States
(12) Patent Application Publication
(10) Pub. No.: US 2008/0227838 A1
(43) Pub. Date: Sep. 18, 2008

(54) Method for neuroprotection with glycoluril derivatives

(75) Inventor: Roman Kamburg, Rishon Le Zion (IL)

Correspondence Address: BROWDY AND NEIMARK, P.L.L.C.;624 NINTH STREET, NW, SUITE 300, WASHINGTON, DC, 20001-5303, US

(73) Assignee: Cereprotec Incorporated, Madison, WI (US)

(21) Appl. No.: 11/723,011

(22) Filed: Mar. 15, 2007

(51) Int. Cl. A61K 31/4188

(52) U.S. Cl. 514/393

(57) ABSTRACT

A method for prevention, inhibition or treatment of neuronal degeneration caused by injury, disease, disorder or condition in the central nervous system (CNS) comprises administering a glycoluril derivative to an individual in need thereof.

### FIELD OF THE INVENTION

The present invention relates to neuroprotective agents and, in particular, to glycoluril derivatives thereof useful in methods for neuroprotection in cases of injuries in the central nervous system (CNS) and in the treatment of neurodegenerative diseases, disorders and conditions.

### BACKGROUND OF THE INVENTION

Damage to the nervous system may result from a traumatic injury, such as penetrating trauma or blunt trauma, or a disease or disorder, including but not limited to Alzheimer's disease, Parkinson's disease, Huntington's disease, amyotrophic lateral sclerosis (ALS), diabetic neuropathy, senile dementia, and ischemia.

Injury to myelinated axons in the CNS often has a devastating outcome in terms of neuronal loss, with accompanying loss of function. Part of the damage occurs immediately, whereas part is delayed and leads to the death of neurons that escaped the direct insult.

Neurodegenerative diseases, disorders and conditions are commonly associated with ongoing neuronal loss in the CNS. Following the loss of neurons caused by primary risk factors, secondary neuronal loss is mediated by self-compounds, such as glutamate, nitric oxide or reactive oxygen species, which exceed their physiological concentrations. These compounds are implicated in various types of neurological disorders and acute CNS injuries. Destructive components common to neurodegenerative diseases have also been identified in autoimmune diseases such as multiple sclerosis. In this disease, myelin damage in the CNS is accompanied by subsequent neuronal loss.

Glycoluril, 2,4,6,8-tetraazabicyclo[3.3.0]octane-3,7-dione, and derivatives thereof, also termed bicyclic bisureas of the octane series, have been described as useful for treating psychic disorders (U.S. Pat. No. 3,966,742; U.S. Pat. No. 4,004,013) and ischemic heart diseases (U.S. Pat. No. 4,571,403). Methods for the synthesis of glycoluril derivatives have also been described (U.S. Pat. No. 3,966,742; U.S. Pat. No. 4,004,013; Chegaev et al., 2001; Kostyanovsky et al., 2002; Kravchenko et al., 2000).

The capability of glycoluril derivatives to exhibit simultaneously both hydrophilic and lipophilic properties is responsible for the physiological action of these compounds: on one hand, they can easily penetrate the body and, on the other hand, readily overcome the hematoencephalitic barrier (Chegaev et al., 2001), also known as a blood-brain-barrier (BBB).

A well known representative of the glycoluril derivatives is the compound named mebicar (2,4,6,8-tetramethyl-2,4,6,8-tetraazabicyclo[3.3.0]-octane-3,7-dione), used as tranquilizer since 1979. Mebicar exhibits extremely low toxicity, does not cause complications and side effects, does not suppress working ability and can be taken under any conditions by people of all ages. Mebicar is not metabolized and leaves the organism within 24 hours.

Another known representative of the glycoluril derivatives is albicar (2,6-diethyl-4,8-dimethyl-2,4,6,8-tetraazabicyclo[3.3.0]-octane-3,7-dione), also described as psychotropically active and candidate for use as tranquilizer (Kostyanovsky et al., 2002).

U.S. Pat. No. 4,004,013 and U.S. Pat. No. 3,966,742 disclose medicinal preparations comprising 2,4,6,8-tetramethyl-2,4,6,8-tetraazabicyclo[3.3.0]-octane-3,7-diones for treating psychic disorders. U.S. Pat. No. 4,571,403 discloses mebicar for treatment of ischemic heart disease.

Previous studies by the present inventor and colleagues have shown that mebicar exerts a protective action on some parameters of body function under the influence of extreme conditions such as stress and hypoxia, within a wide dosage range (100-1000 mg/kg), without affecting the muscle tone, movement coordination or working capacity of tested animals (Zimakova et al., 1980). Mebicar was also shown to change the balance of neuroactive amino acids in the animal's brain by raising the content of gamma-butyric acid (Zimakova et al., 1982), and to exhibit a protective action in experimental arrhythmias (Kamburg and Zimakova, 1982). Mebicar was further shown to produce an antishock action and to normalize acid-base and oxygen homeostasis (Zimakova et al., 1984). Mebicar was also shown to increase the myocardial contractility, to exert a slight effect on the cardiac rhythm and to dilate the peripheral arteries, and the cardiac stimulating effects were attributed to the involvement of mebicar in the myocardial metabolism (Kamburg, 1990).

### SUMMARY OF THE INVENTION

The present invention provides a method for prevention, inhibition or treatment of neuronal degeneration caused by injury, disease, disorder or condition in the central nervous system (CNS), comprising administering to an individual in need thereof an amount effective to treat said injury, disease, disorder or condition of a compound of the formula I:

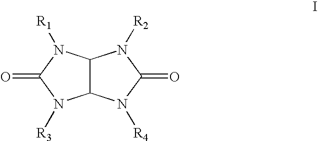

wherein

R₁, R₂ and R₃, the same or different, are H or (C₁-C₆) alkyl;

R₄ is selected from the group consisting of (C₁-C₆) alkyl, —R₅—COOR, —R₅—OH, —R₅—NH—CO—R and —R₅—CO—NH—(CH₂)ₙ—COOR;

R₅ is (C₁-C₆) alkylene;

R is H or (C₁-C₃) alkyl;

n is an integer of 1 to 6;

or an enantiomer or pharmaceutically acceptable salt thereof.

In a preferred embodiment, the compound of formula I is mebicar.

In preferred embodiments, the compounds of formula I are useful for treatment of an injury, disease or disorder selected from the group consisting of: (i) a neurological injury; (ii) brain damage caused by a cardiovascular event; (iii) a cerebrovascular disorder or disease; (iv) a neurodegenerative disease or disorder; and (v) a dementia.

The present invention further relates to an article of manufacture comprising packaging material and a pharmaceutical composition contained within the packaging material, said pharmaceutical composition comprising a compound of formula I or an enantiomer or a pharmaceutically acceptable salt thereof, and said packaging material includes a label that indicates that said agent is therapeutically effective for prevention, inhibition or treatment of neuronal degeneration caused by injury, disease or disorder in the central nervous system (CNS).

### DETAILED DESCRIPTION OF THE INVENTION

The compounds of formula I used in the methods of the present invention are derivatives of glycoluril, also known by its chemical names 2,4,6,8-tetraazabicyclo[3.3.0]-octane-3,7-dione or tetrahydroimidazo[4,5-d]imidazole-2,5(1H,3H)dione. These compounds are termed “bicyclic bisureas (BBU) of the octane series” or “glycoluril derivatives” and these terms are used herein interchangeably to denote a compound of formula I.

As mentioned before, the bicyclic bisurea 2,4,6,8-tetramethyl-2,4,6,8-tetraazabicyclo[3.3.0]-octane-3,7-dione, known as mebicar or mebikar, has been used in medical practice in some countries (mainly in Russia) as a daytime tranquilizer for treatment of psychic disorders (U.S. Pat. No. 4,004,013) and has also been described as useful for treatment of heart ischemic disease (U.S. Pat. No. 4,571,403).

The present inventor has now found that mebicar is useful in an animal model of closed head injury and can be used as a neuroprotective agent for preventing or ameliorating neurological injury response in said models.

The present invention thus provides a method for prevention, inhibition or treatment of neuronal degeneration caused by injury, disease, disorder or condition in the central nervous system (CNS), comprising administering to an individual in need thereof an amount effective to treat said injury, disease, disorder or condition of a compound of the formula I:

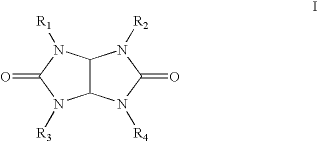

wherein

R₁, R₂ and R₃ the same or different are H or (C₁-C₆) alkyl;

R₄ is selected from the group consisting of: (C₁-C₆) alkyl, —R₅—COOR, —R₅—OH, —R₅—NH—CO—R and —R₅—CO—NH—(CH₂)ₙ—COOR;

R₅ is (C₁-C₆) alkylene;

R is H or (C₁-C₃) alkyl;

n is an integer of 1 to 6;

an enantiomers or a pharmaceutically acceptable salt thereof.

As used herein, the term “(C₁-C₆) alkyl” refers to a straight or branched alkyl and may be, without being limited to, methyl, ethyl, propyl, isopropyl, butyl, isobutyl, pentyl and hexyl, and “(C₁-C₃) alkyl” refers to methyl, ethyl and propyl. As used herein, “(C₁-C₆) alkylene” refers to a straight or branched alkylene radical, and is preferably straight alkylene, such as methylene, ethylene or propylene.

In a preferred embodiment, the method of the invention comprises administration of a compound of formula I wherein R₁ to R₃ each is H or (C₁-C₆) alkyl and R₄ is (C₁-C₆) alkyl. In a more preferred embodiment, the compound is mebicar, wherein R₁ to R₄ each is methyl.

In another preferred embodiment, the method of the invention comprises administration of a compound of formula I wherein R₁ to R₃ each independently is H or (C₁-C₆) alkyl, R₄ is —R₅—COOR, R₅ is straight or branched (C₁-C₆) alkylene and R is H or (C₁-C₃) alkyl. In a more preferred embodiment, R₁ and R₃ each is methyl, R₂ is H and R₄ is —(CH₂)₃COOH.

The glycoluril derivatives of the formula I can be prepared by any of the methods known in the art. For example, mebicar and other tetra-N-alkyl derivatives of glycoluril may be prepared by alkylating glycoluril with dialkylsulphate in a boiling alkaline medium, followed by extraction of the product and its recrystallization (U.S. Pat. No. 3,966,742). Mebicar can be produced also by condensation of N,N-dimethylurea with glioxal in an aqueous medium in the presence of hydrochloric acid, followed by separation of the product. Other methods for the synthesis of glycoluril derivatives of the formula I are described in the literature (see, for example, Kravchenco et al., 2000; Chegaev et al., 2001; Kostyanovsky et al., 2002).

The salts of the compounds of the formula I suitable for use in the present invention are pharmaceutically or physiologically acceptable salts such as the hydrochloride, hydrobromide, sulfate, mesylate, esylate, tosylate, sulfonate, phosphate, or carboxylate salt.

The neuroprotective glycoluril derivatives of formula I may be used according to the present invention in a method for treatment of an injury, disease disorder or condition selected from the group consisting of: (i) a neurological injury; (ii) brain damage caused by a cardiovascular event; (iii) a cerebrovascular disorder or disease; (iv) a neurodegenerative disease or disorder; and (v) a dementia, which comprises administering to an individual in need said derivative of formula I or an enantiomer or a pharmaceutically acceptable salt thereof, in an amount effective to treat said injury, disease, disorder or condition in the individual.

As used herein, the term “an amount effective” refers to an amount effective to ameliorate or alleviate the neurodegeneration caused by said injury, disease, disorder or condition in the individual.

In one embodiment, the compound of formula I is used for treatment of a neurological traumatic injury selected from the group consisting of head trauma injury and spinal cord injury.

In a more preferred embodiment, the patient is treated for an acute neurological traumatic disorder or neurotrauma consisting of a head trauma injury such as blunt trauma, closed head injury and penetrating trauma injury. In these cases, and particularly in closed head injury, it is well known that additional damages or secondary insults follow the main event and it is of importance to administer as soon as possible after the main event a neuroprotective agent that will prevent or inhibit the post-traumatic cell death caused by the main injury. As shown herein in the examples, the glycoluril derivative mebicar was effective in diminishing the degree of brain edema, a secondary insult developed by the test animal after a head trauma model of closed head injury.

In another embodiment, the neurological traumatic injury is spinal cord injury. Also in this type of traumatic injury, it is of importance to protect the neuronal cells from secondary degeneration or secondary insults caused by the main injury in the spinal cord, that lead to partial or complete paralysis of the patient. Administration of a neuroprotective glycoluril derivative according to the invention will prevent or decrease the effects of further neurodegeneration.

Thus, the present invention provides a method for conferring neuroprotection to an individual afflicted with a neurological injury, which comprises administering to said individual a compound of formula I or an enantiomer or a pharmaceutically acceptable salt thereof in an amount effective to ameliorate the neurodegeneration associated with said neurological injury.

In another embodiment of the invention, the glycoluril derivative of formula I is used for treatment of brain damage caused by reduced blood supply due to a cardiovascular event such as cardiac arrest or cardiac surgery.

In a further embodiment of the invention, the glycoluril derivative of formula I is used for treatment of a cerebrovascular disorder or disease such as brain ischemia or stroke. The stroke may be caused by occlusion of cerebral blood vessels, leading to ischemic necrosis of the brain (cerebral infarction) or by rupture of blood vessels resulting in hemorrhage in the brain (hemorrhagic or bleeding stroke). The interruption of blood flow deprives the brain of blood and oxygen and causes the death of brain cells. Impaired blood supply causes immediate loss of neurons at the ischemic site followed by secondary neuronal loss that affects the part of the body it controls. Administration of a neuroprotective glycoluril derivative according to the invention as soon as possible after the event, may prevent the secondary neuronal loss that otherwise will lead to devastating losses in sensory, motor, and cognitive functions, causing paralysis, language and vision and other problems.

In another embodiment, a neuroprotective glycoluril derivative according to the invention may be administered for treatment of a neurological or neurodegenerative disease or disorder such as Alzheimer's disease, Parkinson's disease, facial nerve (Bell's) Palsy, multiple sclerosis, glaucoma, Huntington's chorea, amyotrophic lateral sclerosis and macular degeneration. Along with the main cause, these diseases and disorders are known to cause a degenerative process, e.g. degeneration occurring in either gray or white matter (or both) as a result of the chronic neurodegenerative diseases.

Thus, the present invention further provides a method for conferring neuroprotection to an individual suffering from a neurological or neurodegenerative disease or disorder, which comprises administering to said individual a compound of formula I or an enantiomer or a pharmaceutically acceptable salt thereof in an amount effective to ameliorate the neurodegeneration associated with said neurological or neurodegenerative disease or disorder

In still a further embodiment, a neuroprotective glycoluril derivative according to the invention may be administered for treatment of dementia such as Alzheimer's disease or a non-Alzheimer's dementia selected from the group consisting of senile dementia, Lewy body dementia, vascular dementia and a dementia caused by Parkinson's disease, Huntington's chorea, Creutzfeld-Jacob disease or HIV infection.

The pharmaceutical composition for administration in the method according to the present invention may be in solid, semisolid or liquid form and may further include pharmaceutically acceptable carrier comprising fillers, diluents, and other inert ingredients and excipients.

In one preferred embodiment, the compositions are in the form of tablets, for example, comprising each 0.25-0.5 g of the active ingredient. Also encompassed by the present invention are modified release, sustained release and prolonged release tablets.

The composition can be administered by any suitable route. Thus, mebicar and other glycoluril derivatives of the formula I may be administered orally in the form of tablets or capsules, rectally as suppositories, or injected, e.g. intramuscularly or intravenously, and these and any other suitable form of administration are all encompassed by the present invention.

The dose range of the glycoluril derivative of formula I will depend on the route of administration, the state of the patient and severity of the injury or disease, and will be determined as deemed appropriate by the practitioner. If administered orally as tablets, the daily dosage may be in the range of 0.5-3 g for chronic diseases and 1-5 g, preferably 3-5 g, for acute neurological injuries such as head or spinal cord injury. If administered intravenously for such acute injuries, the daily dosage may be 5-50 ml of a 5-20% solution. This daily dosage may be divided for administration several times a day.

In one embodiment of the invention, the glycoluril compound of formula I is administered to a patient as sole therapy. In another embodiment, it is administered as combined therapy with other known drugs or modalities for treating the injuries, diseases, disorders or conditions in the CNS as recited above.

The invention will now be illustrated by the following non-limiting examples.

### EXAMPLES

### Example 1

### Effect of Mebicar on Cerebral Edema in a Model of Head Trauma

The cerebroprotective effect of mebicar was assessed in a model of head trauma in mice (Beni-Adani et al., 2001). This type of trauma produces brain edema (i.e. increase in water content), breakdown of the blood brain barrier (BBB) and clinical dysfunction. The clinical status of the mice is evaluated 1 and 24 hours after injury along with measurement of the extent of cerebral edema. The neurological deficit, assessed by a set of criteria termed the Neurological Severity Score (NSS), is maximal at 1 hour after the initiation of head trauma. The NSS slowly decreases over time from the initiation of head trauma, with the gradual spontaneous recovery of the mice.

(i) Induction of closed head injury (CHI) in a mice model. Male Sabra mice (strain from Hebrew University of Jerusalem, Israel), weighing 35 to 45 g, were used. CHI was induced under ether anesthesia. A metal rod weighing 333 g was allowed to free fall from a prefixed height (at 3 cm for a mouse weighing 28-32 g) over the exposed skull covering the left hemisphere in the midcoronal plane. The tip of the rod is covered with silicone, and it delivers the impact to the head that is fixed on the bottom plane of the trauma device. Sham-operated mice were anesthetized, their skull exposed, but trauma was not induced. After CHI the mice were assigned to vehicle or mebicar treatment.

(ii) Clinical evaluation of the mice after CHI. Mice were evaluated to define the severity of injury and functional recovery by using a set of clinical criteria. This is a very reliable prognostic tool that correlates with recovery both at the functional and MRI levels, and allows for the evaluation of drug efficacy (Beni-Adani et al., 2001).

For assessment of posttraumatic neurological impairment, a Neurological Severity Score (NSS) previously described, modified and validated, was used (Shapira et al. 1988; Chen et al. 1996; Beni-Adani et al. 2001). The score used herein consists of 10 individual clinical tasks on motor function, alertness, and physiological behavior, whereby one point is given for failure of task performance and zero points for succeeding. A maximal NSS of 10 points indicates severe neurological dysfunction, with failure of all tasks, whereas a score of zero is achieved by healthy uninjured mice. The NSS at 1 hour after trauma reflects the initial severity of injury and is inversely correlated with clinical outcome (Beni-Adani et al. 2001). Evaluation of task performance was done by two investigators who were blinded about the study groups at the time-points 1 h and 24 h after experimental CHI. The ΔNSS, calculated as the difference between NSS at 1 h and the NSS at any later time-point, is a parameter which reflects the degree of spontaneous recovery following brain injury, as described (Chen et al., 1996; Yatsiv et al., 2002; Beni-Adani et al., 2001).

(iii) Evaluation of water content (brain edema). The extent of cerebral edema was evaluated by determining the tissue water content in the injured hemisphere, as previously described (Beni-Adani et al., 2001). Briefly, mice were sacrificed 24 h after injury, the time of peak edema formation, and brain cortical samples of ˜20 mg were cut from the left (traumatized) and right (contralateral) hemispheres, from the site bordering the lesion. The right (non-injured) hemisphere was used as an internal control. Samples were weighed before and after drying in a desiccated oven for 24 h at 95° C. After weighing the “dried” sections, the brain water content was calculated as % H₂O=[(wet weight−dry weight)×100]/wet weight.

(iv) Evaluation of drug effect on brain edema. In order to evaluate the effect of a drug, and to do it in a “clinical-relevant” setting, the drug is given 1 h after the injury, immediately after the initial assessment of NSS. This protocol allows a homogenous distribution of the mice between the drug and vehicle treatments, and ensures similar severity of injury in both groups. NSS is then evaluated again at 24 h after injury, and ΔNSS is calculated. The mice are then sacrificed, their brain removed and cortical segments from the site of injury, and contralaterally, are taken for evaluation of edema, as described above in (iii).

### Results: Mebicar Effect After Brain Trauma

Using the model described above, mebicar in a dose of 750 mg/kg was injected intraperitoneally to mice (10 ml/kg).

The effect of treatment with mebicar on NSS (and on specific motor function) was assessed, as well as the effect on edema and BBB integrity. Spontaneous recovery in clinical function was observed in 2 mice out of 10 (20%), while treatment with mebicar resulted in the recovery of 5 mice out of 9 (55.5%).

TABLE 1
Effect of mebicar on brain edema after brain trauma in mice (M ± SE).

| | 1 | 2 | 3 | 4 | 5 |
|---|---|---|---|---|---|
| | Negative | Positive | Positive | Mebicar | Mebicar |
| | control | Control | Control | treatment | treatment |
| | (normal | (traumatized | (contralateral | (traumatized | (contralateral |
| | rat) | area) | hemisphere) | area) | hemisphere) |
| Water | 78.5 ± 0.5 | 81.9 ± 0.32 | 79.3 ± 0.13 | 80.7 ± 0.41 | 77.9 ± 0.27 |
| content in | | | | | |
| the brain % | | | | | |
| P | | <0.01 vs | <0.05 vs | <0.05 vs control 2 | <0.001 vs |
| | | control 1 | control 1 | | control 3 |

The results, summarized in Table 1, indicate a significant (p<0.05) decrease in the degree of edema developed after head trauma following use of mebicar. About 35% less water accumulated in the traumatized hemisphere of the brain as a result of mebicar treatment to traumatized mice. Mebicar protected absolutely contralateral hemisphere and decreased water content up to normal parameters.

### Example 2

### Effect of Mebicar on Spinal Cord Injury

The neuroprotective effect of mebicar on spinal cord injury can be tested in any suitable animal model, for example, in the model described below.

Acute incomplete spinal cord injury at the low thoracic levels causes an immediate loss of hind limb motor activity that spontaneously recovers within the first 12 days post-injury and stabilizes on deficient movement abilities. The amount of motor function restoration is the sum up effect of the positive recovery from spinal shock and the negative effect of longitudinal and ventral spread of damage. Treatment with a neuroprotective agent may reduce the spread of damage and result in a better recovery in terms of hind limb motor activity.

For testing the effect of mebicar, a contusive injury of the spinal cord is inflicted on anesthetized SPD rats by using a suitable impactor device to drop a 10-g rod from a height of 50 mm onto the exposed laminectomized spinal cord at level T8 (Hauben et al., 2000). The resultant injury is analogous to accidental spinal cord injury in humans. Due to the spinal shock, the motor skills of the rats' hind limbs initially disappears, but recovers with time to reach a steady state of deficient motor activity. The amount of this deficiency caused by the injury can be reduced with adequate neuroprotective treatment. The rats are injected with mebicar or another compound of formula I herein (750 mg/kg), immediately after the injury and 7 days later. The hind limb motor skills of the animals are scored in an open field using the Basso, Beattie, Bresnahan (BBB) locomotor rating scale (Basso et al., 1995), where a score of 0 registers complete paralysis and a score of 2, complete mobility. An untreated group of rats serves as control. Rats treated with mebicar are expected to show a tendency to recover better than untreated rats.

### REFERENCES

Basso, D M, Beattie, M S and Bresnahan, J C. (1995) A sensitive and reliable locomotor rating scale for open field testing in rats, J. Neurotrauma 12:1-21.

Beni-Adani L., Gozes I, Cohen Y, Assaf Y, Steingart R A, Brenneman D E, Eizenberg O, Trembolver V, Shohami E. (2001) A peptide derived from activity-dependent neuroprotective protein (ADNP) ameliorates injury response in closed head injury in mice. J. Pharmacol Exp Ther. 296:57-63.

Chegaev Konstantin Yu, Kravchenko Angelina N, Lebedev Oleg V, Strelenko Yuri A. (2001). New functional glycoluril derivatives. Mendeleev Commun, 11:32-33.

Chen Y., Constantini S., Trembovler V., Weinstock M., and Shohami E. (1996). An experimental model of closed head injury in mice: pathophysiology, histopathology and cognitive deficits. J. Neurotrauma. 13: 557-568.

Hauben, E., Butovsky, O., Nevo, U., Yoles, E., Moalem, G., Agranov, E., Mor, F., Leibowitz-Amit, R., Pevsner, E., Akselrod, S., Neeman, M., Cohen, I. R., & Schwartz, M. (2000) Passive or active immunization with myelin basic protein promotes recovery from spinal cord contusion. J. Neurosci. 20: 6421-6430.

Kamburg R A. (1990) An analysis of the cardiovascular reactions due to mebikar. Farmakol. Toksikol., 53:32-34.

Kamburg R A and Zimakova I E. (1982) Protective effect of the psychtropic preparations diazepam, sodium oxybutyrate and mebikar in experimental arrhythmias. Farmakol. Toksikol, 45:89-91.

Kamburg R A et al. (1986). Results of the experimental study and clinical use of the tranquilizer mebikar. Farmakol. Toksikol, 49:16-19.

Kostyanovsky R G et al. (2002). Chiral drugs via the spontaneous resolution. Mendeleev Commun, 6-8.

Kravchenko A N et al. (2000). New condensation method in the synthesis of bicyclic bisureas. Mendeleev Commun, 10: 27-28.

Shapira, Y., Shohami, E., Sidi, A., Soffer, D., Freeman, S. and Cotev, S. (1988). Experimental closed head injury in rats: mechanical, pathophysiologic, and neurologic properties. Crit. Care Med. 16: 258-265.

Yatsiv I, Morganti-Kossmann M C, Perez D, Dinarello C A, Novick D, Rubinstein M, Otto V I, Rancan M, Kossmann T, Redaelli C A, Trentz O, Shohami E, Stahel P F. (2002). Elevated intracranial IL-18 and evidence of neuroprotective effects of IL-18 binding protein after experimental closed head injury. J. Cereb Blood Flow Metabol 22: 971-978.

Zimakova I E, Kamburg R A, Kirshin S V. (1980) Effect of mebikar on the state of animals under extreme conditions. Farmakol. Toksikol, 43: 368-371.

Zimakova I E, Kirshin S V, Kamburg R A, (1982) Comparative analysis of the behavioral, neurochemical and autonomotropic effects of mebikar and diazepam. Farmakol Toksikol., 45:23-6.

Zimakova I E, Bazarevich Gia, Kamburg R A, Nizamutdinov E Z. (1984) Mechanisms of the antishock action of mebikar. Farmakol. Toksikol, 47: 28-32.

1. A method for prevention, inhibition or treatment of neuronal degeneration caused by injury, disease, disorder or condition in the central nervous system (CNS), comprising administering to an individual in need thereof an amount effective to treat said injury, disease, disorder or condition, of a compound of the formula I: 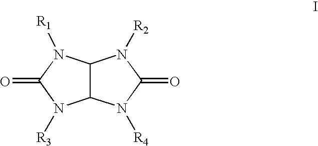
wherein
R₁, R₂ and R₃, the same or different, each is H or (C₁-C₆) alkyl;
R₄ is selected from the group consisting of (C₁-C₆) alkyl, —R₅—COOR, —R₅—OH, —R₅—NH—CO—R and —R₅—CO—NH—(CH₂)ₙ—COOR;
R₅ is (C₁-C₆) alkylene;
R is H or (C₁-C₃) alkyl;
n is an integer of 1 to 6; or an enantiomer thereof or a pharmaceutically acceptable salt thereof.

2. The method according to claim 1 wherein R₁ to R₃ each is H or (C₁-C₆) alkyl and R₄ is (C₁-C₆) alkyl.

3. The method according to claim 2, wherein R₁ to R₄ each is methyl.

4. The method according to claim 1, wherein R₁ to R₃ each is H or (C₁-C₆) alkyl, R₄ is —R₅-—COOR, R₅ is (C₁-C₆) alkylene and R is H or (C₁-C₃) alkyl.

5. The method according to claim 1, wherein R₁ and R₃ each is methyl, R₂ is H and R₄ is —(CH₂)₃COOH.

6. The method according to claim 1, wherein said injury, disease, disorder or condition is selected from the group consisting of: (i) a neurological injury: (ii) brain damage caused by a cardiovascular event; (iii) a cerebrovascular disorder or disease; (iv) a neurodegenerative disease or disorder; and (v) a dementia.

7. The method according to claim 6, wherein said injury is a neurological traumatic injury selected from the group consisting of head trauma injury and spinal cord injury.

8. The method according to claim 7, wherein said head trauma injury is selected from the group consisting of blunt trauma, closed head injury and penetrating trauma injury.

9. The method according to claim 8, wherein said head trauma injury is closed head injury.

10. The method according to claim 6, wherein said injury, disease, disorder or condition is brain damage caused by a cardiovascular event.

11. The method according to claim 10, wherein said cardiovascular event is cardiac arrest or cardiac surgery.

12. The method according to claim 6, wherein said injury, disease, disorder or condition is a cerebrovascular disorder or disease.

13. The method according to claim 12, wherein said cerebrovascular disorder is brain ischemia or stroke.

14. The method according to claim 6, wherein said injury, disease, disorder or condition is a neurodegenerative disease or disorder.

15. The method according to claim 14, wherein said neurodegenerative disease or disorder is selected from the group consisting of Alzheimer's disease, Parkinson's disease, facial nerve (Bell's) palsy, multiple sclerosis, glaucoma, Huntington's chorea, amyotrophic lateral sclerosis and macular degeneration.

16. The method according to claim 6, wherein said injury, disease, disorder or condition is dementia.

17. The method according to claim 16, wherein said dementia is Alzheimer's disease or a non-Alzheimer's dementia selected from the group consisting of senile dementia, Lewy body dementia, vascular dementia and a dementia caused by Parkinson's disease, Huntington's chorea, Creutzfeld-Jacob disease or HIV infection.

18. A method for conferring neuroprotection to an individual afflicted with a neurological injury, which comprises administering to said individual a compound of formula I in claim 1 or an enantiomer or a pharmaceutically acceptable salt thereof in an amount effective to ameliorate the neurodegeneration associated with said neurological injury.

19. The method according to claim 18, wherein said neurological injury is selected from the group consisting of head trauma injury and spinal cord injury.

20. A method for conferring neuroprotection to an individual suffering from a neurological or neurodegenerative disease or disorder, which comprises administering to said individual a compound of formula I in claim 1 or an enantiomer or a pharmaceutically acceptable salt thereof in an amount effective to ameliorate the neurodegeneration associated with said neurological or neurodegenerative disease or disorder.

21. The method according to claim 20, wherein said neurological or neurodegenerative disease or disorder is selected from the group consisting of Alzheimer's disease, Parkinson's disease, facial nerve (Bell's) palsy, multiple sclerosis, glaucoma, Huntington's chorea, amyotrophic lateral sclerosis, macular degeneration and dementia.
